(19) United States
(12) Patent Application Publication
(10) Pub. No.: US 2009/0042736 A1
(43) Pub. Date: Feb. 12, 2009

(54) Biochip and process for the production of a biochip

(76) Inventors: Johan Gerrit Bomer, Haaksbergen (NL); Adrianus Joseph Sprenkels, Lelystad (NL); Colin Ingham, Wateringen (NL); Johannes Epeus Theodoor Van Hylckama Vlieg, Utrecht (NL); Willem Meindert De Vos, Ede (NL); Albert Van de Berg, Nijverdal (NL)

Correspondence Address: NIXON & VANDERHYE, PC, 901 NORTH GLEBE ROAD, 11TH FLOOR, ARLINGTON, VA, 22203, US

(21) Appl. No.: 11/918,570

(22) PCT Filed: Apr. 10, 2006
(86) PCT No.: PCT/NL2006/050078

§ 371 Date: Oct. 16, 2007

(30) Foreign Application Priority Data
Apr. 18, 2005 (EP) 05103080.7

(51) Int. Cl. C40B 30/06; B44C 1/22; C40B 60/12; C12Q 1/04

(52) U.S. Cl. 506/10; 216/12; 435/34; 506/39

(57) ABSTRACT

The invention relates to a biochip (5) comprising a porous support (10), wherein the porous support (10) comprises at least one surface coated with a coating, preferably a polymer coating (20), wherein the coating is patterned with a micro compartments (25) pattern, with the support providing a bottom surface to the compartments and the coating providing edges to the compartments, and wherein the pattern comprises at least 400 compartments per mm2. The biochip (5) can be obtained by a process comprising providing a porous support (10); coating at least one surface of the support with a coating (20); arranging between the coated surface and an ion etching device a shadow mask with a predetermined hole pattern; and ion etching at least part of the coating such that a patterned coating with micro compartments (25) is obtained.

### FIELD OF THE INVENTION

The present invention relates to the field of microbiology. In particular, a biochip comprising a porous support and a micro compartments pattern and process for the production of such biochip are provided. The biochip can for instance be used in a High Throughput Screening (HTS) method for assessing heterogeneity of microorganisms or interaction between microorganisms or for other cellular or growth-based assays. The bio-chip allows the analysis of cells and micro-colonies in situ, with minimal disturbance of cells or microbial communities.

### BACKGROUND ART

The study of inter- or intraspecies diversity (also referred to as heterogeneity) is an important field of interest for ecologists. However, understanding and exploiting the diversity of microorganisms or other cells that can be grown in isolation is also important for advancing the areas of human or animal health or nutrition, food safety, as well as industrial biotechnology, in particular fermentation processes for production of food stuffs, macromolecules with varying degree of polymerization, such as polypeptides, polysaccharides and lipids, as well as other biomaterials, and metabolites. Many food and feed products, including functional foods, and pharmaceutical products contain microorganisms which are either harmless to the subjects ingesting them or have beneficial properties, such as probiotic bacteria. Also, a great variety of microorganisms are used in the production of food ingredients and bulk and fine chemicals. In both pharmaceutical products and food/feed products as well as in industrial applications, generally well-defined microorganisms or populations of microorganisms are used. Preferably, these are organisms, which remain stable and do not vary significantly in phenotype and genotype when grown in culture or when present in the product. Heterogeneity within microbial populations is, therefore, of great interest as are methods to assess this. This not only applies to the industrially used microorganisms but also to those that are undesired and may contaminate a product, such as pathogenic or spoilage microorganisms. Similarly, this applies to microorganisms that are not able to replicate autonomously and need a host to be propagated, such as viruses, bacteriophages and other elements. Finally, heterogeneity is of importance in case new strains are developed for industrial applications, and specifically methods to assess this heterogeneity and select strains with desired traits are of utmost importance.

Heterogeneity in microorganisms has both a genetic and a non-genetic (environment plus noise) component. Micro-organisms are characterized by an extraordinary degree of divergence and heterogeneity, they have representatives in all kingdoms of life and there is a high degree of plasticity and heterogeneity even within a species. Additionally, even within a clonal population, the influence of noise and environment can generate considerable heterogeneity resulting in different states of existence (such as differentiation into spores).

To date no methods are available in the art, which allow the degree of heterogeneity within populations of microorganisms or other cells to be assessed in a rapid and easy way and which consequently allows (a) selection of cells or organisms comprising low or high heterogeneity (e.g. from natural populations, from mutant libraries, from recombinants and the like), (b) the verification of low heterogeneity of single-spore isolates or strains, (c) population and evolutionary studies of microorganisms (including forced or directed evolution) and (d) for cell sorting and the assessment of different cellular states (such as viable but non-culturable organisms or the activities of non- or poorly culturable species) or for detecting or trapping organisms comprising mobile genetic elements (such as viruses, transposons or plasmids).

In addition, there are no high throughput (here abbreviated as HT) methods for analyzing direct or indirect cell-to-cell interactions.

Arrays for use in screening methods have been described in WO 03/102578. The arrays seem to be made by providing wet latex to the support. In this way compartments of 0.5-1 mm can be created. A disadvantage of this technique is that when applied on a porous support, the pores may become substantially blocked, which may make such array unsuitable for high throughput microbiological applications. A further disadvantage is that technology does not allow a high resolution of the compartments.

It is an aspect of the present invention to provide an alternative biochip that can be used in a HTS method. It is further an aspect of the invention to provide a process for the production of such biochip. It is a specifically preferred aspect to provide a process for the production of such biochip with good porosity. It is a further specifically preferred aspect to provide a process for the production of such biochip with a high resolution micro compartment array.

### DEFINITIONS

Herein the term “biochip” refers to a device comprising a porous support, which porous support is preferably flat, and which porous support comprises at least one surface with a micro compartments pattern or structure. Herein, the terms “biochip” and “gridded support” are in an embodiment interchangeable.

The term “micro compartments pattern” refers to a layer on the porous support or a top layer of the porous support which layer has a certain thickness and which layer comprises a number of compartments (preferably at least 400 compartments per mm²). The compartments are notches or cavities in the layer on the porous support or in an embodiment a top layer of the porous support or in a specific embodiment in the porous support itself. The compartments have edges or walls surrounding the compartments, which edges or walls are formed by the layer on the porous support or a top layer of the porous support, or in a specific embodiment by the support itself (when the cavities are in the porous support itself). Such compartments may be round, cubic or rectangular or have other geometries, and have a bottom surface provided by the support and have edges provided by the layer on the support or a top layer of the support. The compartments are present on the biochip as arrays of compartments, preferably in a regular pattern with repeating equal distances between the compartments. The compartments generally have micro or near-micro dimensions, i.e. the compartments generally have a height (depth) of 0.2-1000 μm, preferably 2-100 μm, and a diameter or a width and a length of about 0.5-250 μm, preferably 2-150 μm. Herein the term “grid” is interchangeable with “compartments pattern”. Hence, the term “gridded support” refers to a “support with a compartments pattern”, especially a “support with a micro compartments pattern”.

Herein the term “porous support” refers to a substrate or support with channels as known to the person skilled in the art. The porosity (including pore size) of the support is chosen for allowing compounds, such as nutrients or reporter compounds to diffuse through the pores from underneath the support (“lower surface”) to the bottom of the compartments (“upper surface” or bottom of compartment). At the same time, the pore size must be small enough so that the cells preferably do not pass through the pores. Therefore, in an embodiment, the porosity of the support is chosen such that once a population of cells has been contacted with one surface of the support (the “upper surface”) and the opposite surface (the “lower” surface) of the support is incubated on a medium in order to allow cell growth, cell growth indeed occurs. More specifically, the porous support has pores with pore diameters in the range of 0.005-1 μm, preferably 0.01-0.5 μm, and a porosity of at least 10%, more preferably at least 30%. These porosity values especially refer to the porosity of the areas designed for the culture and/or assay of microorganisms and not other regions of the biochip which are not necessarily porous (like holder parts, markings or other objects that may be present on the biochip).

“Heterogeneity” and “variability” are used herein interchangeably to refer to differences between cells or microorganisms. The term encompasses genetic variation, phenotypic variation and environmental variation.

“Intrinsic heterogeneity” or “natural heterogeneity” refers to the differences or variation already intrinsically present between the cells or a population of cells. For example, the cells may differ in their growth rate when contacted with one particular growth medium. It is noted that external stimuli or factors (e.g. a particular nutrient, stress, heat or a toxic agent) may be needed to reveal the already existing variation between the cells. For example an apparently clonal population of a bacterium may be grown on a solid support and variation in cell length may be observed. The variation however may be moderate as a result of different stages of cell division, unequal partition of molecules in dividing cells, the odd mutation and various other reasons. If this population is stressed in the right way, for example with an antibiotic, a lot more variation in cell length may be seen. In general, subtleties of variation that were already there are possibly revealed. External stimuli or stresses may also lead to repair of damage and mutation and so on that makes the variation larger.

“Microorganism” refers to bacteria, archaea, viruses (including phage), fungi (including yeast species), oomycetes, protozoa, mycoplasmas, algae, microspores, and pollen but also nanobacteria and artificial cells (e.g. by gutting live cells or filling lipid membranes with biomolecules) and/or artificial microorganisms (e.g. hybrid bacteria due to genomic shuffling and mating techniques (e.g. between E. coli and S. typhimurium) and hybrid fungal-bacteria). The term encompasses individual cells (e.g. unicellular microorganisms) or a plurality of cells (e.g. multi-cellular microorganism). A “population of microorganisms” may thus refer to a plurality of cells of a single microorganism (e.g. bacterial cells of one or more species or strains) or to a plurality of cells of two or more different microorganisms, e.g. a mixture of fungal cells and bacterial cells.

“Micro-colony” refers to a small number of cells (two or more), derived from a single cell or from a single microorganism still in close proximity. In this context it is noted that often an inoculum is more than one cell, for example by coincidence or more commonly because more than one cell is joined to another. Therefore micro-colony also refers to colony forming unit (cfu). The formation of a micro-colony involves one or more cell-divisions. In addition “colonies” may also be formed during incubation through changes of a cell without cell division, such as cell growth (enlargement) without cell division or cell differentiation without cell division and colonies may merge or form more complex structures such as bio films. For reasons of clarity these “colonies” will not be referred to as micro-colonies herein, but simply as microorganism(s) or cell(s).

“Interaction(s)” or “cell-to-cell interaction(s)” refers to either direct or indirect interactions between at least two cells (or microorganisms). “Direct interaction” refers to physical contact between the cells. Examples of direct interactions include direct contact of cell walls or membranes (in some cases mediated by specific receptors) or even fusion of these structures or entry of one cell into another, or interactions via cell surface structures such as pili.

“Indirect interaction” refers to indirect contact between the cells, such as through metabolites or signalling compounds or nucleic acids or enzymes or other molecules being secreted by one cell into the medium, wherein only the metabolites come into physical contact with the other cell (or organism). Further, “cell-to-cell interaction(s)” refers to interactions between cells or microorganisms of the same species as well as to interactions between cells or microorganisms of different species including in complex communities such as bio films.

“Reporter compounds” are compounds which assist in the detection of heterogeneity or interactions, such as cell staining dyes, recombinant reporter gene products such as the green fluorescent protein (GFP) or enzymes, etc. Thus, reporter compounds may be either contacted with the cells by external application, for example to the medium underlying the support, or they may be present or induced in, or introduced into, the cells themselves.

“Food-grade” micro-organisms are in particular organisms, which are considered as not harmful, when ingested by a human or animal subject. A “subject” refers herein to a human or non-human animal, in particular a vertebrate, such as but not limited to domestic animals.

“Probiotics” or “probiotic strain(s)” refers to strains of live micro-organisms, preferably bacteria, which have a beneficial effect on the host when ingested (e.g. enterally or by inhalation) by a subject.

The term “comprising” is to be interpreted as specifying the presence of the stated parts, steps or components, but does not exclude the presence of one or more additional parts, steps or components.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

“High-throughput” (HT) as in high throughput screening (HTS) refers to a process designed to perform a large number of assays, including assay(s) on a large number of cells, in an automated or semi-automated fashion. How large this number is will depend on the context of the particular assay, but for example in screening for genetic differences it is desirable to examine millions of cells. In other contexts HTS can be regarded as at least hundreds of thousands of assays or screenings per day but also screening of considerably lower numbers of cells still is considered to be high throughput in the context of this invention. The term “high throughput” also encompasses ultra-high throughput (UHT) “High content” (HC) refers to a variation on HTS in which the amount and quality of the information is of a higher priority, sometimes at the expense of throughput but still dealing with a large number of assays. The term “high throughput” in the context of this invention also encompasses high content and thus HTS also refers to “high content screening” (HCS).

### DETAILED DESCRIPTION OF THE INVENTION

Embodiments of the Product and Process According to the Invention

The biochip according to the invention comprises a porous support, wherein the porous support comprises at least one surface coated with a coating, preferably a polymer coating, wherein the coating is patterned with a micro compartments pattern, with the support providing a bottom surface to the compartments and the coating providing edges to the compartments, and wherein the pattern comprises at least 400 compartments per mm². The biochip according to the invention is obtainable by the process according to the invention. Preferably, the porous support comprises a rigid porous support. The pores are important for allowing compounds, such as nutrients or reporter compounds to diffuse through the pores from underneath the support. At the same time, the pore size must be small enough so that the cells do not pass through the pores. Various supports which are suitable for use in the method are available in the art or can be made using known methods. For example, suitable material for making porous supports may comprise one or more materials selected from the group consisting of acrylic, acrylamide, methylene-bis-acrylamide, dimethylaminopropyl-methacrylamide, styrenemethyl methacrylate copolymers, ethylene/acrylic acid, acrylo-nitrile-butadienestyrene (ABS), ABS/poly-carbonate, ABS/polysulfone, ABS/poly-vinyl chloride, ethylene propylene, ethylene vinyl acetate (EVA), nitrocellulose, poly-carylonitrile (PAN), polyacrylate, polycarbonate, polybutylene terephthalate (PBT), polyethylene terephthalate (PET), polyethylene, polypropylene homopolymer, poly-propylene copolymers, polystyrene, polytetrafluoroethylene (PTFE), fluorinated ethylene-propylene (FEP), ethylene-tetrafluoroethylene (ETFE), perfluoroalkoxy-ethylene (PFA), polyvinyl fluoride (PVF), polyvinylidene fluoride (PVDF), polychloro-trifluoroethylene (PCTFE), polyethylene-chlorotrifluoroethylene (ECTFE), polyvinyl alcohol (PVA), silicon styreneacrylonitrile (SAN), styrene maleic anhydride (SMA), glass and silicon or other etchable materials. Mixtures of two or more of any of these materials may also be used.

In a preferred embodiment the porous supports are made of metal oxides. Such supports, comprising a metal oxide, are already commercially available in the art, e.g. Anopore® inorganic supports (see e.g. WO 99/02266), which are available from Whatman. Anopore is available in various pore sizes, such as 0.02 μm, 0.1 μm and 0.2 μm. Appropriate metal oxide supports have a number of advantages, such as having a high pore density and narrow pore size distribution and they are virtually transparent when wet. Metal oxide supports may be manufactured using methods known in the art, such as electrochemical etching of a metal sheet. Metal oxides include for example oxides of tantalum, titanium, and aluminium, as well as alloys of two or more metal oxides and doped metal oxides and alloys containing metal oxides.

Other suitable supports may include minerals such as zeolites (microporous crystalline solids with well-defined structures (see Handbook of Zeolite Science and Technology, eds. S. M. Auerbach et al. E-ISBN: 0824756126 Pub. Marcel Dekker), rigid fibrous supports or artificially created porous materials. Also hybrids between the materials noted may be suitable. One aspect of the invention is the use of a rigid, porous support as described above, and preferably a metal oxide support, in particular an aluminium oxide support such as Anopore®, for determining the heterogeneity of a population of microorganisms of the same or of different species. Typically such a support is planar.

The supports do not have to have a particular size or shape, i.e. squares, circles, strips of various dimensions may be used. Particular preferred is an area equivalent to a 96-well plate footprint as commonly used in the art. Other preferred dimensions include that of a typical microscope slide.

In one embodiment, the support comprises a grid (compartments pattern or compartments structure), comprising at least 400, 500, 800, 1000, 2000, 4000, 8000, 10000, or more compartments per mm². The grid can be deposited on the porous support (e.g. Anopore) using printing, photolithography, sputtering or other methods. The polymer used can be polyimide or other high precision polymer (SU8) or a photo-activated film or a metal such as gold (vide infra).

The grid can be as small as 5 microns high (deep) and 5 wide (or even less) leaving compartments of as little as only 1 or 2 microns. This will allow around 10,000 compartments per mm² (i.e. 1 million per cm and over 100 million on a 96-well plate-sized area). Such a grid will essentially harbour only 1 or a few bacteria per compartment. Preferably, the compartments have a height (depth) of 0.2-1000 μm, preferably 2-100 μm and a diameter or a width and length of about 0.5-250 μm, preferably 2-150 μm. This means that the coating preferably has a thickness of 1-1000 μm. Particularly feasible are compartments of approximately 20 by 20 microns (bottom surface area or compartment area dimensions), or approximately 30 by 30 microns, which results in a support having more than about 2000, respectively more than about 1000 compartments per mm². Preferably, the compartment area (of the bottom surface) of the compartments is in the range of 1 μm²-50,000 μm², preferably 4 μm²-40,000 μm, even more preferably 20 μm²-20,000 μm². Preferably, the edges of the compartments have a height in the range of 0.2-1000 μm, preferably 2-100 μm. In a specific embodiment, the pattern comprises at least 1,000 compartments, more preferably 10,000 compartments per mm². In an even more specific embodiment, the pattern comprises at least 100,000 compartments per mm². The wall thickness between two adjacent compartments, or the distance between two adjacent compartments is preferably at least 0.5 μm, more preferably at least 1 μm, even more preferably at least 2 μm. Since the walls between adjacent compartments are not necessarily perpendicular to the bottom of the compartments and may have a slope, the wall thickness herein refers to the thickness of the wall measured at the bottoms of adjacent compartments. Since the walls between compartments may be structured, the wall thickness refers to the average thickness.

As mentioned above, the grid or micro compartments structure can be produced in a number of ways. A preferred process for the production of the biochip, wherein the biochip comprises a porous support with a micro compartments pattern for growing cultures of micro organisms, comprises:
a. providing a porous support;
b. coating at least one surface of the support with a coating, preferably a polymer coating, preferably having a thickness of 0.2-1000 μm;
d. arranging between the coated surface and an ion etching device, preferably a reactive ion etching device (RIE), a shadow mask with a predetermined hole pattern; and
e. ion etching at least part of the coating such that a patterned coating with micro compartments is obtained.

The porous support has been described above. The process of coating at least one surface of the support with a coating having a thickness of 0.2-1000 μm, preferably 2-100 μm, can be performed with techniques known in the art. Preferably the coating is a polymer, which is provided to the porous support. Such coating may be bonded to the porous support by using heat and pressure or may be glued to the support, with glues known to the person skilled in the art. In a specific embodiment, the coating comprises one or more materials selected from the group consisting of Teflon, Polyester, polyimide, SU8, thermoplastic polymer such as PMMA (polymethyl methacrylate), POM (polyoxymethylene), PC (polycarbonate), PCDF (polychlorinated dibenzofuran) and PSU (polysulfone), ABS (acrylonitrile butadiene styrene), PVC, (polyvinyl chloride), polypropylene, polyethylene, acrylic, celluloid, polystyrene cellulose acetate, rubber and polydimethylsiloxane (PDMS). In an embodiment, non-polymeric materials may also be suitable, such as metals or other materials. Preferably, the polymer coating or other material comprises a laminate. Such laminate preferably includes an adhesion layer and a polymer layer. The laminate may be arranged to the porous support by adhering the adhesion layer to the support. In this way a stack is obtained comprising a porous support, an adhesion layer and a polymer layer, wherein the latter two layers are comprised in the laminate. The micro compartments pattern is than provided in the laminate (vide infra).

Preferably, the coating layer (polymer, especially the laminate) acts as a physical barrier to the spread of microorganisms and as such is effectively bond to the porous support. This implies that the coating layer is preferably arranged on the support in such a way that no gaps are created (i.e. gaps between compartments or gaps between coating layer and porous support), or only gaps are created smaller than about 0.2 μm. Preferably, gaps larger than 0.2 μm are absent. Gaps that lead to a break in the continuity of a barrier between two compartments or by poor adhesion to the porous support are avoided or may only be present when smaller than about 0.2 μm. Further, the layer thickness of the coating layer is chosen that at an appropriate thickness to create a barrier of the right height. In addition, the coating layer is preferably not toxic or detrimental to microbial culture or related assays such as determination of the activity of a microbial enzyme. Preferably, the coating layer is sterilisable along with the rest of the chip by at least one of a heat, a steam (autoclaving), an irradiation or a chemical sterilization method. Such sterilization methods are known in the art. Further, the coating layer is removable from specific areas (for forming the growth compartments of the micro compartments pattern) of the coating layer by the process of the invention, especially etching such as reactive ion etching (RIE). In addition, preferably the coating layer is durable (for instance not degraded by micro organisms, not degraded by commonly used chemicals (such as ethanol, dimethylformamide, dimethylosulfoxime, methanol, acids, alkaline reagents and ideally other solvents including chloroform and acetone), not degradable by day light or microbial action, and not degrading during a reasonable storage period of, for instance, about several years.

It may further be preferable that the coating layer has a relatively low adhesion for micro organisms so that inoculations do not adhere to the walls (where they may be unculturable or promote contamination between compartments. It may further be preferable that the coating layer generates a relatively low signal in terms of background for a predetermined detection method, particularly does not show autofluorescence or only shows weak autofluorescence under irradiation with for instance UV light. It may further be desirable that the physical properties (especially hydrophobicity) should reduce the ability of the compartments to act as capillaries (if they do, they will fill with culture media transforming the invention from culture on a solid support to a form of liquid culture).

Although the ion etching method according to the process of the invention does not imply the use of laminates comprising an adhesion layer and a photoresist layer (i.e. a photo resist polymer for use in photolithography), since the method does not use photolithography, it appear that such laminates may advantageously be applied in the process of the invention. In a specific embodiment, the polymer coating for use in the process of the invention comprises a dry film photopolymer resist.

Whereas it surprisingly appears that photo etching techniques may lead to blocking of the pores of for instance anopore, the ion etching technique used in the process of the present invention advantageously does not provide blocked pores.

It appears that the support porosity of the support provided with a micro compartments pattern is preferably ≧90% of the initial support, not yet provided with the micro compartments pattern. Even more preferably, the support porosity of the support provided with a micro compartments pattern is ≧95%, preferably ≧99%, of the initial support, not yet provided with the micro compartments pattern.

The permeability of chips provided by the process of the invention using ion etching methods can be assessed in a number of ways. For instance, the biochip can be placed on a filter paper disk on a sintered glass support. Excess area around the chip is blocked with for instance parafilm (so air flow is through chip). Then, drops of water are dripped on the biochip. By applying vacuum to the biochip water may be pulled through the biochip. Another method is to spread the upper surface of the biochip with a microorganism such as Lactobacillus plantarum WCFS1, with for instance an average of 100 colony forming units per compartment. Medium below the biochip is stained with a dye, for instance with 10 micromolar Syto9 dye (Invitrogen). Immediately stain from beneath penetrates through the pores and stains bacteria in unblocked compartments. The bacteria in unblocked compartments will stain with the dye, which may for instance be detected by fluorescence microscopy and which can be counted. Another way may be to inoculate the upper surface of the biochip with microorganism such as Lactobacillus plantarum WCFS1 with at least 10 colony forming units per compartment. The biochip is placed on MRS agar (Oxoid) and cultured anaerobically overnight at 37° C. Stain from below with for instance Syto9 (see above) and compartments that have supported growth can be assessed by for instance fluorescence microscopy (generally full of bacteria indicating >6 rounds of cell division assuming exponential growth).

In this way, a biochip is provided wherein ≧90%, preferably ≧95%, even more preferably ≧98%, yet even more preferably ≧99% of the micro compartments facilitate growth of microorganisms within the compartments. In order to estimate the number of micro compartments facilitating growth (which is related to the porosity of the biochip) a person skilled can choose a microorganism, for instance mentioned above, and grow the microorganism in the micro compartments, as for instance described above. The number of compartments that show growth or staining should be at least 90% of the compartments. When for instance prior art methods would be used, like the method of WO 03/102578, even when compartments with the small dimensions according to the invention (at least 400 compartments per mm², preferably at least 1,000 compartments per mm², even more preferably at least 10,000 compartments per mm²) would be manageable at all, such compartments would have blocked pores, not facilitating growth (due to blocking of the pores by latex) etc.

Further, the ion etching technique for etching away the polymer advantageously provides a micro compartments pattern in the coating without substantially affecting the adhesion of the polymer coating. It appears that when photolithographic etching techniques (photolithography) are used, the polymer coating after processing does not adhere well, whereas the polymer coating when using the process of the invention does adhere well after processing.

Especially suitable polymer coatings for use in the process of the invention comprise laminar plates that are designed to be used as dry film photoresists such as ordyl dry film photoresists from Tokyo OHKA Industrial or Elga Europe, for instance Ordyl SY 300 (like 314).

As mentioned above, the coating has a coating thickness of about 1-1000 μm. For instance, laminar 5000 dry film photopolymers of Eternal such as Laminar 5025, 5032, 5038, 5050 and 5075 have a coating or layer thickness of about 25-75 μm. Then a ion etching device is provided and between the coated surface and the ion etching device a shadow mask with a predetermined hole pattern is arranged. Subsequently, at least part of the coating, preferably a polymer coating, is ion etched such that a patterned coating with micro compartments is obtained. Such techniques are known in the art. RIE (reactive ion etching) is a preferred ion etching technique and refers to chemically enhanced “ion bundle etching” (IBE), methods known to the person skilled in the art. IBE is a pure physical bombardment of the specimen with fast moving ions to remove layers from the specimen. This process is relatively slow and therefore less suitable. By adding a chemical compound such as CF₄ or SF₆ in the etching chamber the etching rate is increased and the process is called RIE. Etching is continued until at predetermined positions where the compartments have to be arranged, the coating is etched away and anopore starts being treated by the ions of the ion etching method. In this way, compartments are provided wherein the support provides a bottom surface to the compartments and the coating provides edges to the compartments. Therefore, the invention provides highly miniaturized flow and growth chambers for microbial culture and other biochips at relatively low cost based structures built onto the porous support, especially anopore.

Such shadow mask mentioned above for use in the process according to the invention is also an aspect of the invention. Techniques for producing such shadow masks are known in the art. In the process of the invention, the shadow mask is arranged to provide a patterned polymer comprising at least 400 compartments per mm². In a specific embodiment, the shadow mask is arranged to provide a patterned polymer with compartment areas in the range of 20 μm²-20,000 μm². Preferably, the shadow mask is arranged to provide a patterned polymer with wall thicknesses between adjacent compartments of at least 0.5 μm, more preferably at least 1 μm, even more preferably at least 2 μm. These parameters concerning number of compartments, compartment area of the compartments and wall thicknesses, respectively, imply that the shadow mask is designed to provide such patterns, respectively, by ion etching techniques, especially deep reactive ion etching techniques, preferably according to the process of the invention.

In a specific embodiment, instead of a polymer coating, a metal or other material coating is applied. Hence, in a specific embodiment, a process for the production of a biochip comprising a porous support with a micro compartments pattern for growing cultures of micro organisms is provided, the process comprising:
a. providing a porous support;
b. coating at least one surface of the support with a metal coating, preferably having a thickness of 1-1000 μm;
c. arranging between the metal coated surface and an ion etching device, preferably a reactive ion etching device (RIE), a shadow mask with a predetermined hole pattern;
d. ion etching at least part of the metal coating such that a patterned metal coating with micro compartments is obtained.

Therefore, a biochip is provided, the biochip comprising a porous support, wherein the porous support comprises at least one surface coated with a metal coating, wherein the metal coating is patterned with a micro compartments pattern, with the support providing a bottom surface to the compartments and the metal coating providing edges to the compartments, and wherein the pattern comprises at least 400 compartments per mm². The term “metal” and “coating” also refer to a combination of two or more metals and a combination of two or more coatings, respectively. A metal that can be used is for instance Au (gold), Pt (platinum) and Ti (titanium).Instead of a metal, also other materials may be applied, such as for instance alloys, metal nitrides or materials, etc.

In yet another embodiment, the support comprises silicon, especially porous silicon, silicon nitride or silicon dioxide. Porous silicon substrates can be provided in an embodiment by etching one or both sides of a silicon substrate (including silicon nitride or silicon dioxide) resulting in compartmented structures with underlying thinner parts that can be made porous in a third etching step, for instance an electrochemical etch step.

Hence, in a specific embodiment, a process for the production of a biochip comprising a porous support with a micro compartments pattern for growing cultures of micro organisms is provided, the process comprising:
a. providing a support (preferably a wafer), preferably a silicon substrate (like a silicon wafer);
b. arranging between a first surface of the support and an ion etching device, preferably a reactive ion etching device (RIE) a shadow mask with a predetermined hole pattern;
c. ion etching at least part of the first surface such that a patterned support with micro compartments is obtained;
d. etching the support at least at one or more positions where the thickness of the support between the first surface and a second surface opposite of the first surface has been reduced due to the presence of one or more micro compartments, thereby providing an at least partial porous support.

In this way, a porous support may be provided wherein the compartments are processed into the support, thus without the use of the polymer or a metal coating layer. In such embodiment, the compartments are integrated into the porous support (in fact porous made support), and the compartment wall(s) are provided by the support. Thereby, a biochip comprising a porous support, wherein the porous support is patterned with a micro compartments pattern, with the support providing edges to the compartments, and wherein the pattern comprises at least 400 compartments per mm². Preferably, the bottom surface area of the compartments is in the range of 20 μm²-20,000 μm² and preferably the edges of the compartments have a height in the range of 0.2-1000 μm. Especially preferred are silicon, silica and silicon nitride supports, such as wafers, although also other materials may be used as support.

The porous support may thus only be porous below the compartments. Further, the porosity may be introduced to the support before or after providing the compartments.

For any embodiment of the biochip described above, the support comprises a grid (compartments pattern or compartments structure), comprising at least 400, preferably 500, more preferably 800, even more preferably 1000, yet even more preferably 2000, more preferentially 4000, even more preferentially 8000, yet even more preferentially 10000, or more (especially 100000) compartments per mm.

The biochip of the invention is obtainable by the processes described according to the invention.

Embodiments of Methods According to the Invention

In one embodiment of the invention a HTP method for determining the heterogeneity of a population of microorganisms, is provided.

The method comprising the steps of:
(a) contacting the microorganisms with a rigid, porous support,
(b) incubating the support on a medium to allow cell growth, cell division (micro-colony formation) and/or cell differentiation of the microorganisms,
(c) determining the heterogeneity of the microorganisms or micro-colonies (preferably without disturbing, or with minimally disturbing, the location of the microorganisms on the support), and
(d) optionally repeating steps (b) and (c) one or more times, and
(e) optionally selecting one or more microorganisms.

In particular the method of the invention allows determining the intrinsic heterogeneity of a population of microorganisms.

The focus of the present method is thus on measuring heterogeneity rather than on determining a magnitude of response. As discussed above for WO 03/102578, in the prior art a HTS involves treating a bacterial population with an antibiotic and seeing if it kills the target bacterium. For the prior art many aspects of heterogeneity are a problem—it is noise that makes the assay more difficult and less reliable. The present invention thus is not concerned with the absolute effect of an external factor such as for example an antibiotic, but the focus is on how an external factor brings the diversity and stability of the population to light.

To carry out the method of the invention initially a plurality of cells or microorganisms are provided, which are to be analysed. The starting population of cells may vary, depending on the aim of the analysis.

For example, the starting population may be a mixture of microorganisms found in a natural sample, such as a soil sample, a water sample, an air sample, a urine sample and the like. Alternatively, the sample may be a man-made composition, such as a food or drink, or a composition which is to be used in the preparation of a food or drink (such as a starter culture to be used in the preparation of fermented products, such as yoghurts, beer, etc.). Similarly, libraries of mutant or recombinant microorganisms (e.g. mutant or recombinant bacteria) may be analysed. Also, single strains or isolates commonly used in research or in the preparation of pharmaceutical or nutritional compositions may be analysed, in order to determine whether these are homogeneous (i.e. there is low heterogeneity between the cells derived from a single starting cell or colony) and stable. In principle any starting population of cells or microorganisms may be used.

The starting population may initially be grown, for example in liquid culture, to increase the number of cells. For example, if heterogeneity of a single spore isolate of a fungus is to be determined, the single spore isolate may first be grown to provide a plurality of cells derived from it. Likewise, the starting population may be purified or partially purified using methods known in the art (for example by filtration or centrifugation or with a fluorescent activated cell sorter) prior to contacting the population with the support. The removal of debris and other non-cellular components may be desirable for accurate detection.

The starting cells or microorganisms may be of a single species or of a mixture of species. Similarly, if the starting microorganism is of a single species, it may be of a single strain (e.g. single clone or strain) or it may be a mixture of strains. Examples of species that may be analyzed are (plant or animal) pathogen species. For example human or animal pathogens include such bacteria as Legionella pneumophila, Listeria monocytogenes, Pseudomonas aeruginosa, pathogenic E. coli strains, Salmonella ssp., Klebsiella spp., Hafnia alvei, Haemophilus influenzae, Proteus spp. Serratia spp. Shigella spp. Vibrio spp., Bacillus species including B. anthracis and B. cereus, Campylobacter jejuni, Yersinia spp. Clostridium perfringens, Enterococcal species such as E. faecalis Neisseria meningitides or N. gonhorrhea, Streptococcus ssp. including S. pyogenes and S. pneumoniae, Staphylococcal species such as S. aureus including MRSA, Mycobacterium tuberculosis, Enterobacter (e.g. Enterobacter cloacae) etc. Additionally pathogenic fungi, such as yeasts, e.g. Candida species including C. albicans, C. krusei and C. tropicalis, and filamentous fungi such as Aspergillus fumigatus or Penicillium marneffei including dermatophytes such as Trichophyton rubrum. Additionally, free-living protozoans such as pathogenic free-living amoeba may also be analysed or protozoans carrying bacterial pathogens such as Legionella. Plant pathogens include for example Pseudomonas species (e.g. Pseudomonas solanacearum), Xylella fastidiosa; Ralstonia solanacearum, Xanthomonas campestris, Erwinia amylovora Fusarium species, Phytophthora infestans, Leptosphaeria species powdery mildews (Ascomycota) and rusts (Basidiomycota). etc.

In a preferred embodiment compositions comprising one or more species or strains of food-grade microorganisms and/or probiotic microorganisms such as but not limited to species of the genera Lactobacillus, Streptococcus, Lactococcus, Oenococcus, Leuconostoc, Pediococcus, Carnobacterium, Psychrobacter, Pseudomonas, Propionibacterium, Enterococcus and Bifidobacterium, Brevibacterium, Corynebacterium are used. Yeast species, such as Saccharomyces cerevisiae (baker's yeast or brewer's yeast) or S. pastorianum (lager yeast) may also be used as are fungi such as Aspergillus or Rhizopus spp.

Additionally, other micro-organisms are envisaged, for example those used in industrial processes such as the production of enzymes (e.g. in biological washing powders from organisms such as Bacillus subtilis) or thermophilic Archeaea (e.g. Sulfolobus sulotaricus) or other extremophiles or with uses in bioremediation or yeasts and fungi such as Hhansenula, Pichia, Aspergillus etc. Strains involved in bioleaching for metal release from ore may also be used, such as Geobacter sulfureducens and Pseudomonas isachenkovii.

Examples of starting populations of microorganisms will be given further below, as the aim of the analysis determines which cells to start with. For example if the aim is to test whether a certain strain is homogeneous and stable, one starts with a plurality of cells of this strain.

In step (a) the microorganisms, or the composition comprising these, are contacted with a rigid, porous support.

The cells may be applied to one or to both surfaces of the support. In a preferred embodiment the microorganisms are contacted with the upper surface of the support, while the lower surface of the support is contacted with (solid, liquid or semi-solid) medium. “Contacting” can be achieved by various methods, such as (but not limited to) dipping, spreading, spraying or pipeting of a composition comprising the microorganisms onto one or both surfaces.

The cell density on the support can be adapted as desired, by preparing compositions (e.g. liquid suspensions in sterile water) comprising a suitable cell concentration. Also, dilution series of cell suspensions may be used as inoculums for a series of supports. Although for most applications it is generally preferred that cells are distributed so that they are separated from one another in order to allow micro-colonies being formed (preferably arising from one or more cell divisions of a single cell), higher concentrations may also be suitable. For example, cells may be in close proximity or in contact with one another. For some applications, the densities may be sufficiently high to allow bio films (i.e. cell monolayers or multiple layers) to be formed following incubation. Suitable concentration of support-inoculum may thus result in cell densities of e.g. 100 to 2000 cells per mm² of support, or more. Obviously, suitable cell densities may differ from one microorganism to another, and may again be different for mixtures of organism. Likewise, the aim of the analysis (heterogeneity or cell interaction) will also influence the choice of cell density. A skilled person can easily determine the optimal cell density and inoculum concentration for a desired analysis.

Once the population of cells has been contacted with the support, the support is in step (b) incubated on a medium in order to allow cell growth, cell differentiation and/or micro-colony formation. The medium used may comprise one or more of the following: nutrients, minerals, other compounds such as chemical inducers or inhibitors of cellular processes, antibiotics or toxins, proteins or peptides, carbohydrates or nucleic acids, compounds that may influence cellular interaction or adhesion, enzyme substrates, reporter molecules, etc. Preferably the medium is homogenous with respect to the support, although a medium comprising gradients of one or more compounds along the support is also envisaged for certain applications. For example, a 2D gradient of the two most important end-metabolites in fermentation could be used to test that a production strain was stable under the range of conditions encountered during a fermentation. Also, a gradient could be used to define the concentration of two signalling compounds that trigger a cell:cell interaction, such as a mating.

The medium may be solid, liquid or semi-solid. For example a simple agar medium, suitable for maintaining cell viability, growth, cell division and/or differentiation, may be used. The pH may be adapted, depending on the organisms being tested. In one embodiment the medium is a growth medium, suitable for culturing the microorganism(s). Such media are well known in the art. The medium may also be one which allows selective growth of one or more species of microorganisms or induces particular changes (e.g. spore formation or germination).

The incubation conditions (and the medium) may also vary, depending on the microorganisms and the phenotypic characteristics which are to be analyzed. Thus, incubation temperature(s) chosen may vary, incubation period(s) may vary, humidity may vary, aerobic or anaerobic conditions may be used (e.g. for facultative anaerobes anaerobic conditions are required), etc. Preferably, when bacteria are incubated the incubation time is at least about 20 minutes, which allows micro-colony formation, e.g. comprising on average 2, 3, 4, 5, 6, 7, 8, or more cells per micro-colony. Incubation times may range from 20 minutes to several hours, up to about eight hours.

In one embodiment prior to and/or during step (a) and/or during step (b) the heterogeneity within the population is revealed or amplified by submitting the microorganisms to external stimuli (or factors) such as heat, cold, salt, toxins, antibiotics or other antimicrobial compounds, osmotic stress, infectious agens such as viruses etc.

Heterogeneity thus refers to difference between cells or microorganisms. Differences can be generated by genetic means (specified by nucleic acids, possibly other heritable mechanism such as covalent modification of stable or replicated molecules), by differences in environment or experience or by chance, such as unequal partition of rare molecules during cell-division. Usually such differences come to light as a phenotypic characteristic. “Phenotypic characteristics” (or “phenotypes”) refer herein to any feature or combination of features (whether macroscopic, microscopic, molecular, biochemical, physiological) of the cells which is to be measured or assessed and which indicate the degree of heterogeneity between the cells or between (or within) micro-colonies, such as (but in no way limiting): cell or micro-colony sizes, shape(s), textures, ability to retain stains or dyes and/or colors; growth rate, viability, differentiation or behaviour including motility, nucleic acid distribution; gene expression; protein (enzyme) production, changes in cell wall (including septation), capsule, membranes or other layer(s) surrounding the cell or structures protruding from the cell such as flagella or pili, metabolite production (e.g. folic acid levels) or aspects of energy transduction or consequences of metabolism such as changes in pH, changes in organelle or vesicle structure, secreted products including hormones and signalling peptides or quorum sensing autoinducers or nucleic acids or enzymes; mRNA levels (transcription of one or more genes); DNA or mRNA fingerprints; protein compositions, levels or activities; responsiveness to environmental factors; presence/absence or transfer of mobile genetic elements (transposons, viruses, plasmids, etc.); the (degree of) direct or indirect interaction between micro-organisms such as predation, formation of complex multicellular structures or communities (such as bio films) that may be of the same or of different species, competition for nutrients, action of bacterocidins, release of signalling compounds that result in the formation of complex communities, adhesion, close cooperation between organisms including sharing of energy metabolism, etc.

The phenotypic characteristics may be determined qualitatively (e.g. presence or absence of a feature) or quantitatively (e.g. length of cells).

Step (c) involves the determination of the heterogeneity or of the interaction between the cells, microorganism or micro-colonies. This step involves the scoring of one or more phenotypic characteristics, as defined above. For example, if the intra- or inter-micro-colony heterogeneity in terms of cell length or morphology is to be analyzed, the length of a large number of cells is measured and statistical analysis of the data is used to determine the heterogeneity of the population (see Examples). More examples will be given herein below. Obviously, the methods used for detecting the heterogeneity (i.e. for assessing one or more phenotypes) may vary, depending on the characteristics to be scored.

The support surface may be examined through a light microscope (e.g. light-, fluorescent-, confocal-) or scanning electron microscopy or by surface plasmon resonance, conductivity, enzyme assays, mass spectrometry, etc.. Thus “image analysis” of the support may be carried out (“image analysis” refers to the surface examination of the support surface, either by eye, e.g. through a microscope, or by a camera or laser scanner or other apparatus scanning the support surface, which then in turn may produce images or any other output data, such as counts, etc.). Thus, in a preferred embodiment the support surface is analysed directly using laser scanning, especially rapid laser scanning methods, whereby the support surface is scanned to detect the microorganims or the reporter compound. Such scanners are available in the art such as the ChemScan®RDI (Chemunex InCell Analyser 3000 (RTS Life Science International/Amersham Biosciences).

In one embodiment a rapid (in situ) staining method is used. In this method the support is transferred to a medium comprising one or more reporter compounds, such as (fluorescent or luminescent) dyes which stain e.g. living (or dead) cells, or particular cellular components (e.g. nucleic acids, etc). The reporter compounds are able to diffuse through the pores of the support and thereby rapidly come into contact with the cells, with minimal disruption of the location of the cells on the support. If the reporter compound is already present or produced in the cells (e.g. GFP protein), it need not to be brought into contact with the cells. However, in certain detection methods the reporter compound is only released or developed in or by the cells upon contact with another compound (e.g. an enzyme, which cleaves a compound present in the cells, thereby causing the cell to emit light). In such indirect detection methods the other compound(s) needed are also referred to as reporter compounds herein, even though these are not the compounds detected directly.

The rapid staining method does, therefore, not necessarily require cell fixation, which has the advantages that the cellular distribution is retained and that cells or sub-cellular components are not damaged. Artefacts are thereby avoided.

In one embodiment the reporter compound is a dye or luminescent compound (e.g. a fluorogenic or chromogenic compound) or a mixture of several compounds, such as Fluo-3, Fluo-4, and Ca-dyes such as e. g. Calcium Green-1, Syto dyes (Invitrogen), fluorescein isothiocyanate, rhodamine, malachite green, Oregon green, Texas Red, Congo red, SybrGreen, phycoerythrin, allophycocyanin, 6-carboxyfluorescein (6-FAM), 2′,7′dimethoxy-4′,5′-dichloro-6-carboxyfluorescein (JOE), 6-carboxy X-rhodamine (ROX), 6-carboxy-2′,4′,7′,4,7-hexachlorofluorescein (HEX), 5-carboxyfluorescein (5-FAM), N,N,N′,N′tetramethyl-6-carboxyrhodamine (TAMRA), cyanine dyes (e. g.Cy5, Cy3), BODIPY dyes (e. g. BODIPY630/650, Alexa542, etc), green fluorescent protein (GFP or eGFP), blue fluorescent protein (BFP), yellow fluorescent protein (YFP), red fluorescent protein (RFP). Fluorescent nano-particles such as quantum dots may also be employed. Advanced uses of fluorescence dyes as above in such techniques as FRET or in vivo tracking of molecules is also envisaged.

For determining cell viability, viability labels such as for example Fluorassure® or calcein/calcein-AM based fluorescent compounds may be used (e.g. obtainable from Sigma; esterases within viable cells cleave calcein-AM to produce calcein, which is then detected and is directly proportional to the number of viable cells). Esterase substrates include cFDA cell-tracker compounds (Molecular Probes). The medium is, for example, contacted with a (fluorogenic or chromogenic) substrate, which is taken up and converted by a viable cell to a fluorochrome or chromophore, which can then be detected. Viability or metabolic activity may also be detected by pH or membrane potential-sensitive fluorescent agents (e.g. tetrazolium-based dyes).

Reporter compounds may also be nucleic acids including modified analogues thereof or radioactively labelled molecules, peptides, proteins, and antibodies or antibody fragments, enzyme substrates, etc. For example, the support may be contacted with labelled antibodies (e.g. monoclonal antibodies raised against a specific antigen). The antibody, attached to the cell may be detected directly (by detection of the label attached thereto), or indirectly by using e.g. another labelled antibody directed against the specific itself. Labelled cells may then be detected by e.g. fluorescence microscopy or laser scanning. It may be advantageous to apply this particular method in combination with cell fixation (vide infra).

Means for detecting the reporter compound are known in the art. Thus, for example, radiolabels may be detected using photographic film or scintillation counters, fluorescent markers may be detected using a photo detector to detect emitted illumination. Enzymatic labels are typically detected by providing the enzyme with an enzyme substrate and detecting the reaction product produced by the action of the enzyme on the substrate, and colorimetric labels are detected by simply visualizing the coloured label. Further detection means are for example (micro-) calorimetry and (light)-microscopy.

Optionally, in one embodiment the cells may be fixed to the support prior to being analysed further. Cell fixation methods are known in the art. Also, cells of interest may be picked off the support and analysed ex situ (directly or after sub-culturing or replica plating), e.g. in PCR reactions, ELISA assays, nucleic acid hybridization, staining, etc. Preferably such detection methods are automated and high throughput.

The detection methods are thus not limited and include molecular-, histochemical-, microscopic-, enzymatic-analysis, such as for example DNA sequencing, PCR analysis, nucleic acid hybridization, immunological tests, enzymatic assays, microscopy, etc., all as known in the art and as for example described in Sambrook et al. (1989), Sambrook and Russel (2001), Ausubel et al. (1994), Brown (1998), Brock, Smith and Madigan (1984). Protocols, methods, kits and media are available commercially from a range of suppliers, such as Bio-Rad Laboratories, S.A., Sigma-Aldrich and many others.

As already mentioned steps (b) and (c) may be carried out repeatedly. For example, the incubation conditions and/or the medium may be varied between repeats or different detection assays may be carried out. Also, one or more cells may be selected from the support for further analysis or growth. For example, cells with particular properties may be selected (e.g. a cell producing more folic acids than other cells, or a cell which grows faster than other cells).

In one embodiment the method comprises such a grid, with nutrients being supplied through the support pores from below. The microorganisms are spread plated onto the support and suction or electrophoresis is used to draw the cells into the compartments. Bacterial contamination on the surface can be prevented using antibacterial surfaces, antibacterial light or other method if it is necessary to remove or kill them. It is important that the material used to create the walls or barriers does not itself act as a capillary or the test areas will flood with nutrients allowing escape of cells.

When microorganisms, e.g. bacteria, are grown they can achieve colony heights of at least 1 mm or more. In this embodiment the only way for cells in the compartment to grow is to multiply upwards, out of the compartments. When very small compartment-sizes are used this happens after only a few rounds of cell division. The cells grown out of the compartments can be detected quickly and easily, for example by laser scanning or conductivity methods as a rapid and extraordinarily high throughput system with applications in industrial microbiology, antimicrobial screening and other areas. For example, the different test areas can be supplied with different antimicrobial compounds and viability counts performed.

### DESCRIPTION OF THE FIGURES

FIGS. 1a-c schematically depict side and top views of a biochip 5 according to the invention. Biochip 5 may have a width b1, for instance 7.5 mm and a length L1 of 35 mm. The thickness h2 of anopore support 10 may be about 60 μm. FIG. 1a shows a support 10, which is at least partially porous (i.e. porous regions), with (polymer) coating 20 and arranged above (or on top of polymer coating 20) a shadow mask 30, with predetermined openings 31, which are arranged to provide a patterned (polymer) coating according to the invention. This means that the shadow mask 30 is arranged between an ion etching device (not shown) arranged to generate ions for etching polymer coating 20 (or a metal or other material) and polymer coating 20 (or a metal or other material). The top of material 20 is indicated with reference number 33. This top 33 is also indicated as first surface or top surface of biochip 5. The bottom surface of second surface of biochip 5 is indicated with reference number 11. Support 10 may for instance be anopore. By an ion etching process (especially RIE), wherein ions are impinging (schematically indicated with reference number 40) on coating 20, biochip 5 is obtained, as shown in FIGS. 1b and 1c. Here, coating 20 contains compartments 25, which have a bottom surface 21a formed by substrate 10 and which have side walls formed by coating 20. The compartments comprise a top opening, due to the etching process, as is clear from the figures. The interface between support 10 and coating 20 is indicated with reference number 21. This interface 21 will also provide the bottom surface 21a to the compartments 25, although it is not excluded that during RIE processing, also a top layer of anopore is etched away. In general however, this will be kept to a minimum. Hence, in general height hi of compartments 25 is identical or substantially identical to the height of coating 20. Height hi is for instance 15 μm. The compartment area is the area of compartments 25 provided by bottom surface 21a, which is the area of compartments 25 enclosed by the compartment walls (i.e. coating 20). Distances between adjacent compartments 25 are indicated with reference symbol d2 and may be for instance 1 μm (this is also indicated with barrier width). Diffusion 13 of micro organisms is prevented by using coatings 20 that adhere well to support 10. Diffusion 12 of nutrients from the bottom surface to the top surface of support 10 is possible due to the porosity of support 10. FIG. 1c shows compartments 25 with a square bottom surface (see also FIGS. 2a-2), but rectangular, oval, round or other shaped compartments may also be applied. The support 10 is porous. This means that support 10 is either provided as porous material, such as anopore, or is made porous after providing the grid structure with compartments 25. For instance, support 10 may be etched at least at one or more positions where the thickness of the support 10 and coating between the first surface 33 and a second surface 11 opposite of the first surface 33 has been reduced due to the presence of one or more micro compartments 25, thereby providing an at least partial porous support (at positions 51 below such compartments 25).

Support 10 is at least porous below compartments 25. A region below compartment 25, i.e. the region in support 10 between bottom surface 21a and second surface 11, is indicated with reference number 51. At least the regions below compartments 25 are porous and have a porosity as described above.

Coating 20 may be a polymer as described above, or a laminate like a dry film photoresist. However, herein coating 20 may also comprise a metal or other material.

FIGS. 2a-b show SEM measurements of biochips made according to example 5.

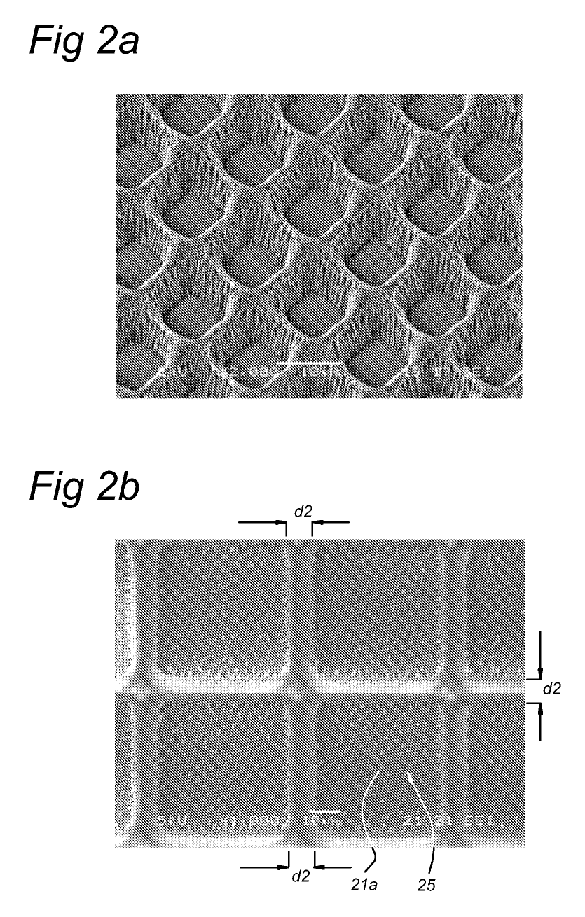

As shown in the embodiment of FIGS. 2a-2b, square compartments 25 are provided into coating 20, the coating 20 providing side walls (with wall thickness d2) and the support 10 providing a bottom surface 21a, with a top opening.

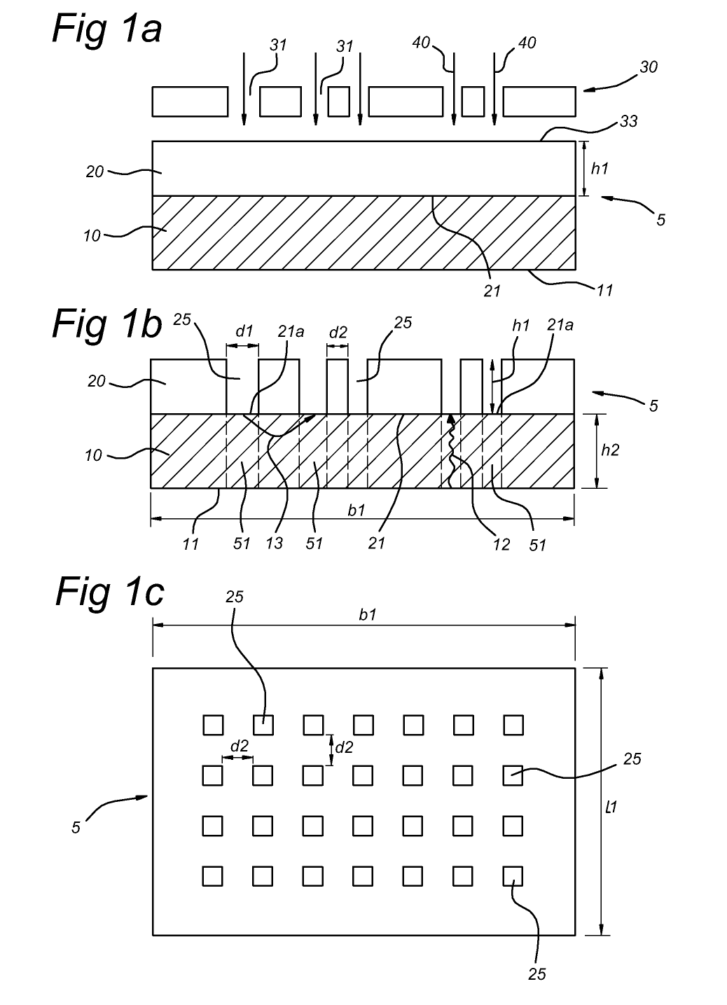

In a specific embodiment, the compartments 25 are processed into the support 10 itself. Referring to FIGS. 1a and 1b, material 20 and support 10 are than one material, such as a silicon dioxide wafer. In this embodiment, support 10/20 is etched to provide compartments 25. Since the support 10/20 may not be porous before providing the grid structure or compartment structure, a further etching to provide porosity may be applied. At least at one or more positions 51 where the thickness (h1+h1) of the support between the first surface 33 and a second surface 11 opposite of the first surface 33 has been reduced due to the presence of one or more micro compartments 25, thereby providing an at least partial porous support, which is indicated with regions 51. Hence, the phrase “providing a porous support” includes the situation wherein a non-porous or substantially non-porous support is provided, and after (or during) providing the compartment structure, the support is made porous, at least at those positions below compartments 25, as indicated in the figures. Here, the porosity is as described above.

### EXAMPLES

The following experiments show the development of microbial cell heterogeneity during growth to a microcolony on anopore with imaging in situ.

### Example 1

### Heterogeneity in Cell Size and Nucleic Acid Distribution Material and Methods

Escherichia coli (strain 2613) was inoculated on sterile Anopore (0.2 micron pore size, 60 microns thick, 8 mm×36 mm strips) at a density of 2000 cells per mm² of area.

The strip was incubated at 37° C. for 2 hours by placing it on 2TY agar to allow formation of micro-colonies on the upper surface of the Anopore by division of cells from the inoculum.

The Anopore was then moved to a microscope slide covered in a thin film of solidified low-melting-point agar containing the nucleic acid-binding dye Syto9 (Invitrogen).

This method allowed rapid staining of the cells on the Anopore with minimal disturbance of the cells on the surface by the dye passing upwards though the pores and entering the cells. After 10 minutes, the micro-colonies are imaged by means of a BX41 fluorescent microscope equipped with Fluorotar lenses (Olympus, x50 objective used). Data was captured using a Kappa CCD camera.

Even in micro-colonies as small as 8 cells, considerable heterogeneity in cell size and nucleic acid distribution was observed. Cells are typically 2-3 microns long.

### Example 2

### Heterogeneity in Cell Length and Cell Growth Material and Methods

Strain WCFS 1 of Lactobacillus plantarum was inoculated on anopore (as in Example 1).

The anopore was incubated at 37° C. for 5 hours on MRS agar under anaerobic conditions to allow formation of micro-colonies.

Electron microscopy (Cryo-SEM) was used to image the resulting micro-colonies directly on the anopore surface.

The length of the cells in each of three distinctly separated micro-colonies was calculated using the cell-analysis program ImageJ (NIH). As a control, repeated measurements of a randomly chosen cell were made to assess the variability of the measuring technique.

Both intra-colony heterogeneity and inter-colony heterogeneity in cell length was observed.

### Example 3

### Interaction between Micro-Colonies

The two colonies are Enterobacter cloacae grown on Anopore® for 4 hours on Mueller-Hinton agar at 37° C. then stained from below using a mixture of Syto9 dye and Hexidium Iodide. The inoculum was stressed by its environment prior to inoculation on Anopore®, normally Hexidium Iodide penetrates this species poorly but here heterogeneity has been observed with the 2-dye system. In this case no bias indicating any interaction was observed.

### Example 4

### Gridded support

Growth compartments were created on Anopore using a photosensitive (photoresist) film. Initially, the film (Laminar 5000, Shipley UK) was used to laminate the upper surface of the Anopore. Photolithographic techniques were then employed, using a series of masks, to direct the selective and permanent polymerisation of the photoresist and the removal of unpolymerised material (Shipley Laminar 5000 technical data sheet PI 102701). The end result was an Anopore chip. A single chip was based around a 36×8 mm strip of anopore to create over 20,000 growth compartments of c. 100 microns across. A series of washes (methanol, ethanol, acetone and sterile water) was used to remove residues and to ensure that the Anopore beneath the compartments was not blocked. After a final ethanol wash to ensure sterility, and air drying, the chip was ready for use.

To use, an Anopore chip was placed in an agar plate of MRS medium (Oxoid) and spread plate with cells of Lactobacillus plantarum strain WCFS 1 at an average density of 20 cells per compartment. Chips were incubated under anaerobic conditions for 10 hours at 37° C. then stained with Syto9 dye and imaged as described previously, or by scanning electron microscopy.

Many compartments now contained thousands to tens of thousands of cells, indicating that multiplication of the inoculum has occurred. The walls segregate the cells in different compartments creating a highly multiplexed testing and screening environment.

### Example 5

### Gridded Support Produced with RIE

A microbial culture biochip was engineered that maintains and improves the advantages of separation of organism on a planar surface, but which addresses many of the limitations of the Petri dish. The material chosen was a highly porous ceramic (Anopore). Anopore has previously been shown to be a good growth and imaging support for bacteria and fungi. The limited change in volume of Anopore with wetting or temperature changes is an additional advantage in micro-engineering this material. The micro engineering of Anopore to create channels for molecular analysis and growth compartments with a density of 200 cm⁻² is known. Here, the microbial culture possibilities is extended with a novel approach that creates culture areas of up to or more than 350,000 cm⁻² (i.e. up to or more than 3500 mm⁻²) a number highly appropriate for HTS and other applications.

Construction of a substrate holder: In order to handle the fragile anopore substrates in the clean room equipment, a substrate holder to fix the substrates is used. A P-type <1−0−0> silicon wafer was covered with 200 nm silicon nitride. A pattern with the dimensions of the anopore substrates was etched in the silicon nitride on the front side of the wafer with Electrotech PF340 reactive ion etcher (3 min at 10 mTorr, 75W, 25 sccm CHF₃/5 sccm O₂), using a usual photolithography process. After stripping the photoresist in an oxygen plasma, the cavities were etched in KOH (1 hour in 25% KOH at 75° C.) until 55 μm depth.

Cleaning and lamination of Anopore: Anopore strips (35×7.5 mm², 60 μm thick, pore size c. 0.2 μm) were put in the substrate holder and cleaned with oxygen plasma using an Electrotech PF340 reactive ion etcher (5 min at 10 mTorr, 100W, 20 sccm O₂). Ordyl 314 foil (supplier Elga Europe S.r.l., Milano; http://www.elgaeurope.it/) was used to laminate the entire upper surface of Anopore strips using a heated roller (Laminator GBC 3500) at 110° C. Polymerisation of the laminate was initiated by exposure to 365 nm UV-light in a KarlSuss mask aligner for 30 seconds; then by baking for 1 hour at 150° C. to complete the curing process.

Ordyl SY 314 belongs to the Ordyl SY 300 series, which laminates may be used in the invention. Ordyl SY 300 is a solvent type permanent dry film for special MEMS applications. It is a negative-working photopolymer and is designed to be applied with hot roll lamination. Ordyl SY 300 is capable of resolving patterns down to 40 μm. Ordyl SY 300 coatings can be processed with CFC free chemicals. Advantageously, Ordyl SY 300 shows compatibility with biological fluids. It has a strong adhesion to different materials (glass, silicon, epoxy resin, polymers, etc). It has a resist thickness of 15-50 μm. It can be hot rolled at temperatures of about 90-105° C., at a lamination pressure of about 1.0-2.5 bar and with a lamination speed of 0.5-2.0 m/min. Similar dry film photoresist may also be used, although the photoresist properties are not relevant for the process of the invention (see above).

Construction of a shadow mask: The mask pattern was etched with DRIE (Deep Reactive Ion Etching) in the front side of a <1−0−0> p-type silicon wafer (previously cleaned by 1% HF acid for 1 min and subsequent 5 min oxygen plasma in a barrel etcher (Tepla300E) at 500W and 1.3 mbar O₂). Photolithography was done by spinning HDMS adhesion promoter for 20 sec 4000 rpm and subsequently 20 sec 4000 rpm OiR908-17 resist on the front side of the silicon wafer. After alignment with the mask 3.7 sec exposure on an Electrovision (EVG620) Mask aligner, developing and then baking for 5 min at 90° C. on a hot plate. DRIE of the compartments was done using an Adixen100SE I-speeder deep Si etcher for 12 minutes by a Bosch process. Next the backside of the mask was locally etched with KOH (5 hour 20 min in 25% KOH at 75° C.) with a structured silicon nitride layer as mask, until a 20 μm thick membrane with precisely defined holes resulted. The silicon nitride mask was stripped in HF. Finally, a protective layer of aluminium oxide (150 nm thick) was sputtered to reduce etching and back-sputtering of the mask during its use.

Reactive ion etching of laminated Anopore: The silicon shadow mask was aligned with a substrate holder containing 16 laminated strips of Anopore and etched with PlasmaTherm 790: parallel plate reactive ion etcher (20 mTorr, 500W, 40/4 sccm O₂/CHF₃) for 25 min (5 treatments of 5 min, with intervals of 3 min to cool-down). Next the anopore chips were cut out of the substrate holder by means of a scalpel. After treatment, the completed chips were washed and sterilized; twice in distilled water and twice in 96% (v/v) ethanol and stored in sterile tubes ready for use.

Below, some characteristics of biochip embodiments are given:

| | Compartment | Barrier | Total Area | Compartments |
|---|---|---|---|---|
| embodiment | Width | Width (μm) | (μm²) | cm⁻² |
| 1 | 100 | 10 | 12100 | 8264 |
| 2 | 40 | 10 | 2500 | 40000 |
| 3 | 20 | 10 | 900 | 111111 |
| 4 | 20 | 20 | 1600 | 62500 |
| 5 | 7 | 10 | 289 | 346021 |

| | Compartments: | Compartments: | Cells per | Division to |
|---|---|---|---|---|
| embodiment | strip | plate | monolayer | monoloyer |
| 1 | 23802 | 1586777 | 6667 | 12 |
| 2 | 115200 | 7680000 | 1067 | 10 |
| 3 | 320000 | 21333333 | 267 | 8 |
| 4 | 180000 | 12000000 | 267 | 7 |
| 5 | 996540 | 66435986 | 33 | 5 |

| embodiment | Comp Area (μm²) | % Area Open |
|---|---|---|
| 1 | 10000 | 83 |
| 2 | 1600 | 64 |
| 3 | 400 | 44 |
| 4 | 400 | 25 |
| 5 | 49 | 17 |

Culture of microorganisms: Anopore strips or microbial chips were placed on MRS agar (Oxoid, NL) and inoculated with Lactobacillus plantarum strain WCFS1 and incubated under anaerobic conditions at 37° C. in Anaercult A minibags (Merck). After incubation, the bacteria were stained with Syto9 (Invitrogen, NL) in situ through the pores of the chip from beneath.

Image capture and analysis: Chips were imaged directly (without immersion oil or cover-slip) using an Olympus BX41 epifluorescence microscope equipped with UmPlan F1 objective lenses and U-MWIBA and U-M41007 filters (Olympus, Japan). Image capture used a Kappa CCD camera (Kappa, Germany). TIFF files of 8-bit images were analysed quantitatively using ImageJ software to implement background correction, median filtration, conversion to a binary image and measurement of colony or cell size. Images were merged and displayed using Photoshop 8.0 CS (Adobe). Scanning electron microscopy was as previously described.

Chip manufacture: RIE was used to selectively remove an acrylic polymer laminated on the surface of Anopore. The compartments formed were extremely precisely defined and it was possible to remove all material from the Anopore by precisely optimising the etching time—too little time resulted in incomplete removal of the laminate whilst excessive etching time resulted in redeposition of material back onto the Anopore from the mask, effectively a process of back-sputtering, as will be clear to the person skilled in the art.

Growth of L. plantarum WCFS1 on Anopore: Growth of L. planturum on sterile anopore strips, placed on MRS medium and incubated anaerobically, could be detected within 1 hour of inoculation of a stationary phase culture and imaging Syto 9 stained cells in situ. Measuring the parameters of cell length, cell number and micro colony area, comparing time 0 with 1 hour a significant increase (P<0.001 Mann Whitney U test, n=200 all samples for all three parameters) was observed. Growth of L. plantarum WCFS1 was rapid and, after a lag-phase, exponential with a μmax of 0.76 similar to a value of 0.68 observed in anaerobic liquid batch culture, both at 30° C.

Growth of L. plantarum within compartments on Anopore chips: Effective growth and segregation of organisms was seen in compartments with a culture area from 150×150 to 7×7 μm. Even with overnight incubation clear demarcation was seen between adjacent culture compartments suggesting that the wall was an effective barrier to the spread of micro-organisms between adjacent areas. Inoculation by spread-plating suggested that a dilution series could be accurately counted and that spatial biases were not excessive.

In comparison, photolithographic etching (according to example 4) created compartments of good morphology but during creation or during later steps (such as ethanol sterilisation) relatively weak adherence of the material to the Anopore was apparent, resulting in loss of the structures or cross-contamination between compartments. In contrast, detachment of the material was never a problem with the RIE and there was very little evidence of the spread of microorganisms between compartments (as would be revealed by an atypical distribution, particularly with a low inoculum, one compartment effectively spreading into adjacent compartments). It further appeared that the biochip with compartments provided according the process of the invention including reactive ion etching, had good porosity, whereas the wet etched biochip according to example 4 often had blocked pores.

Hence, within the region of a compartments then the porosity of the laminated and etched Anopore or other porous substrate is close to the porosity of Anopore that has never been laminated (i.e. the porosity under the compartments is ≧90% of the original porosity), for instance tested by dye penetration for example, using a fluorogenic dye such as Syto 9 added from below to stain organisms spread but not grown on the upper surface or by the growth test, wherein a high percentage of the intended compartments (≧-90%) should be sufficiently porous to support microbial growth at an acceptable rate.

### Example 6

### Gridded Support Produced with RIE

Here another example is given of the micro-engineering of a microbial growth format capable of culturing millions of bacterial or fungal samples. The base material upon which the organisms are grown is a sheet of a porous aluminium oxide (sold as Anopore). Anopore is a rigid and exceptionally porous (such as 2×109 pores cm⁻², pore diameter of 0.2 μm) inorganic, planar material. Anopore is extremely flat with low background fluorescence and is translucent when wet: making it appropriate as a cellular imaging substrate and also amenable for micro-engineering. The chip formats are flexible, permit rapid exchange of nutrients and other materials through the pores and do not desiccate. The planar surface permits imaging by fluorescence microscopy and other techniques. Microbial culture is possible on strips of this ceramic with the nutrients supplied from beneath the Anopore to organisms on the upper surface. Growth can be monitored at the single cell and microcolony level using a variety of (fluorescence) microscopy techniques.

By laminating the Anopore with a thin sheet of an acrylic polymer, and selective removal by dry reactive ion etching, directed by means of a shadow mask, discrete growth compartments were constructed on the surface of the Anopore (see also FIG. 2a). This is a novel approach which is very effective at creating structures on a porous surface. The compartments are 7-150 μm squares separated by barriers 10-20 μm high and wide. This results in a (highly regular array of miniaturized culture areas capable of supporting growth combined with in situ imaging) at densities ranging from 8200 to 360,000 growth areas cm⁻¹. This allows the creation of chips with millions of culture areas: well within the range required for high throughput screening. Culture, containment of microbial growth within discrete areas has been demonstrated for Lactobacillus plantarum strain WCFS 1 and other micro-organisms.

Additional tools for the transfer and inoculation of organisms and functionalities that modify the properties of these chips, are being created. The prospects for application of these chips as diagnostic tools, for cell counting and screening will be shown.

The process and product of the invention can be applied in a number of technical fields. It is a relatively low cost method, which is further flexible in terms of material choice and a relatively simple process compared to other patterning techniques. Specific applications include:

HTS/HCS—creation of highly-multiplexed screening formats for cellular assays—for example industrial strain improvement or screening for antimicrobials.

Microbial Diagnostics—creation of chips and simpler formats for antibiotic sensitivity testing, organism ID.

Environmental monitoring—Detection of organisms, for example in food processing or pharmaceutical or cosmetic manufacture. Detection and enumeration or difficult to culture organisms.

General Microbiology —Rapid enumeration and detection or identification of organisms.

Other Anopore Based Assays or Devices—e.g. Modification of anopore into multiwell plates and molecular bioassay chips (e.g. channelling reaction components through a particular section of anopore). Possible utility in non-biological applications (anopore is a popular material in nanotechnology).

1. A process for the production of a biochip comprising a porous support with a micro compartments pattern for growing cultures of micro organisms, comprising:
a. providing a porous support;
b. coating at least one surface of the support with a coating, preferably a polymer coating;
c. arranging between the coated surface and an ion etching device a shadow mask with a predetermined hole pattern; and
d. ion etching at least part of the coating such that a patterned coating with micro compartments is obtained.

2. The process according to claim 1, wherein the support provides a bottom surface to the compartments and the coating provides edges to the compartments, and wherein the coating preferably has a thickness of 0.2-1000 μm.

3. The process according to claim 1, wherein the shadow mask is arranged to provide a patterned coating comprising at least 400 compartments per mm².

4. The process according to claim 1, wherein the ion etching device is a reactive ion etching device (RIE).

5. The process according to claim 1, wherein the support comprises anopore.

6. The process according to claim 1, shadow mask is arranged to provide a patterned coating with compartment areas in the range of 20 μm²-20,000 μm².

7. A biochip comprising a porous support, wherein the porous support comprises at least one surface coated with a coating, wherein the coating is patterned with a micro compartments pattern, with the support providing a bottom surface to the compartments and the coating providing edges to the compartments, and wherein the pattern comprises at least 400 compartments per mm².

8. The biochip according to claim 7, wherein the bottom surface area of the compartments is in the range of 20 μm²-20,000 μm² and wherein the edges of the compartments have a height in the range of 0.2-1000 μm.

9. The biochip according to claim 7, wherein the pattern comprises at least 10,000 compartments per mm².

10. The biochip according to claim 7, wherein the pattern comprises at least 100,000 compartments per mm².

11. The biochip according to claim 7, wherein the porous support comprises one or more materials selected from the group consisting of acrylic, acrylamide, methylene-bis-acrylamide, dimethylaminopropylmethacrylamide, styrenemethyl methacrylate copolymers, ethylene/acrylic acid, acrylonitrile-butadienestyrene (ABS), ABS/poly-carbonate, ABS/polysulfone, ABS/polyvinyl chloride, ethylene propylene, ethylene vinyl acetate (EVA), nitrocellulose, polycarylonitrile (PAN), polyacrylate, polycarbonate, polybutylene terephthalate (PBT), poly-ethylene terephthalate (PET), polyethylene, polypropylene homopolymer, polypropylene copolymers, polystyrene, polytetrafluoroethylene (PTFE), fluorinated ethylene-propylene (FEP), ethylene-tetrafluoroethylene (ETFE), perfluoroalkoxy-ethylene (PFA), polyvinyl fluoride (PVF), polyvinylidene fluoride (PVDF), poly-chlorotrifluoroethylene (PCTFE), polyethylene-chlorotrifluoroethylene (ECTFE), polyvinyl alcohol (PVA), silicon styreneacrylonitrile (SAN), styrene maleic anhydride (SMA), glass and silicon.

12. The biochip according to claim 7, wherein the support comprises a metal oxide, preferably anopore.

13. The biochip according to claim 7, wherein the polymer coating comprises one or more materials selected from the group consisting of Teflon, Polyester, polyimide, SU8, thermoplastic polymer such as PMMA (polymethyl methacrylate), POM (polyoxymethylene), PC (polycarbonate), PCDF (polychlorinated dibenzofuran) and PSU (polysulfone), ABS (acrylonitrile butadiene styrene), PVC, (polyvinyl chloride), polypropylene, polyethylene, acrylic, celluloid, polystyrene cellulose acetate, rubber and polydimethylsiloxane (PDMS).

14. The biochip according to claim 7, wherein the coating comprises a laminate.

15. The biochip according to claim 7, wherein the laminate comprises an adhesion layer and a photoresist layer.

16. The biochip according to claim 7, wherein the coating comprises a dry film photopolymer resist.

17. The biochip according to claim 7, wherein ≧90% of micro compartments facilitate growth of micro organisms within the compartments.

18. The biochip according to claim 7, wherein the porous support has pores with pore diameters in the range of 0.005-1 μm, preferably 0.01-0.5 μm, and a porosity of at least 10%, more preferably at least 30%.

19. A shadow mask for use in the method according to claim 1.

20. A high throughput method for determining heterogeneity within a population of microorganisms comprising the steps of:
a. contacting the microorganisms with a porous support,
b. incubating the support on a medium to allow micro-colony formation, cell growth and/or cell differentiation of the microorganisms,
c. determining the heterogeneity of the microorganisms or micro-colonies, and
d. optionally repeating steps (b) and (c) one or more times, and
e. optionally selecting one or more microorganisms.

21. The method according to claim 20, wherein the porous support is a biochip comprising at least one surface coated with a coating, wherein the coating is patterned with a micro compartments pattern, with the support providing a bottom surface to the compartments and the coating providing edges to the compartments, and wherein the pattern comprises at least 400 compartments per mm².
